(19) United States
(12) Patent Application Publication
(10) Pub. No.: US 2010/0158890 A1
(43) Pub. Date: Jun. 24, 2010

(54) USE OF THROMBIN MUTANTS TO INHIBIT THE ANTICOAGULATION EFFECT OF THROMBIN INHIBITORS

(76) Inventors: Kenichi Tanaka, Atlanta, GA (US); Enrico Di Cera, Ladue, MO (US); Andras Gruber, Portland, OR (US); Stephen Raymond Hanson, Beaverton, OR (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/282,695

(22) PCT Filed: Mar. 15, 2007
(86) PCT No.: PCT/US07/64081

§ 371 Date: Oct. 6, 2009

Related U.S. Application Data
(60) Provisional application No. 60/782,648, filed on Mar. 15, 2006.

(51) Int. Cl. A61K 38/48; G01N 33/53; A61P 7/04

(52) U.S. Cl. 424/94.64; 435/7.1

(57) ABSTRACT

The present invention provides methods for inhibiting the anticoagulation effect of a thrombin inhibitor in a patient in need thereof comprising administration of a therapeutically effective amount of a variant prothrombin or thrombin that is capable of binding the thrombin inhibitor and that has reduced procoagulant activity. Variant prothrombins or thrombins of use in the methods of the present invention include thrombin mutants W215A, W215A/E217A, or variants thereof in which the amino acids at positions 215 and/or 217 are alanine. Methods are also provided in which the thrombin mutants are administered with an additional active agent, particularly hemostatic agents such as activated factor VII or activated prothrombin complex concentrate. In one embodiment of the invention, the methods are useful in the treatment of patients in which a direct thrombin inhibitor has been administered, particularly argatroban. The present invention further provides a method for quantifying the concentration of an anticoagulant in the plasma or whole blood of a patient using a variant prothrombin or thrombin titration assay.

### FEDERALLY SPONSORED RESEARCH OR DEVELOPMENT

This invention was made with government support under grant numbers HL49413, HL58141, and HL73813 awarded by the National Institutes of Health. The United States government has certain rights in the invention.

### FIELD OF THE INVENTION

The invention relates to methods for inhibiting the anticoagulation effect of thrombin inhibitors through the use of thrombin mutants, in particular thrombin mutants W215A/E217A or W215A. The invention also relates to methods of use for thrombin mutants to quantify the concentration of thrombin inhibitors in the plasma or whole blood of a patient.

### BACKGROUND OF THE INVENTION

Anticoagulants such as heparin and coumarin are frequently used in the treatment and prevention of thromboembolic diseases and to prevent blood clotting during blood transfusions and following surgical procedures. However, administration of such drugs can lead to complications that include heparin-induced thrombocytopenia-thrombosis and coumarin-induced skin necrosis; complications that can ultimately result in limb loss. It is therefore highly desirable when administering an anticoagulant to a patient to have an antidote on hand that can reverse the effects of that particular anticoagulant in the event that complications occur. Such complications include hemorrhagic side effects in wounds from surgical incision as well as in the vascular regions of the peritoneum, the pleura, the pericardium and the pia mater.

Anticoagulant agents may generally be divided into indirect thrombin inhibitors and direct thrombin inhibitors (DTIs). Indirect thrombin inhibitors such as heparin and dermatan inactivate thrombin by catalyzing the activation of endogenous thrombin inhibitors such as antithrombin (AT) or heparin cofactor II (HCII). By contrast, the effects of direct thrombin inhibitors such as hirudin are mediated by binding directly to thrombin.

With respect to indirect thrombin inhibitors, heparin is often used due to the advantage that its anticoagulation effects are rapidly reversed using protamine sulfate. Nevertheless, protamine administration may cause catastrophic cardiovascular collapses, although rare (Morel et al. (1987) Anesthesiology, 66:597-604; Weiss et al. (1989) New England Journal of Medicine, 320:886-892; Panos et al. (2003) European Journal of Cardio-Thoracic Surgery, 24:325-327) and protamine-induced hemodynamic instability is associated with adverse post-operative outcomes (Kimmel et al. (2002) Anesthesia & Analgesia, 94:1402-1408; Welsby et al. (2005) Anesthesiology, 102:308-314). There is currently no safe alternative heparin reversal for those with protamine allergy.

Due to limitations and risks associated with conventional anticoagulants, clinical uses of DTIs are rapidly evolving. All DTIs are potent antithrombotic agents, and major hemorrhagic risks are in the range of 0.7-1.9%, comparable to or less than unfractionated heparin (Di Nisio et al. (2005) New England Journal of Medicine, 353:1028-1040). Currently available DTIs include argatroban (Novastan®), bivalirudin (Angiomax®), lepirudin (Refludan®), and, in Europe, ximelagatran (Exanta®) (Di Nisio et al. (2005) New England Journal of Medicine, 353:1028-1040). The main indication for intravenous DTIs is heparin-induced thrombocytopenia-thrombosis, but other indications including elective percutaneous coronary interventions, and coronary bypass surgery are currently being evaluated (Lincoff et al. (2004) JAMA, 292:696-703; Merry et al. (2004) Annals of Thoracic Surgery, 77:925-931). Ximelagatran is the first oral agent for prophylaxis of deep vein thromboses.

However, the use of specific DTIs may be limited due to the unavailability of antidotes and lack of reliable laboratory monitoring (Warkentin et al. (2005) Thrombosis & Haemostasis, 94:958-964). Underdosing anticoagulant may result in uncontrolled thrombin generation and/or consumptive coagulopathy, whereas overdosing anticoagulant leads to serious bleeding diatheses. Clotting tests are particularly vulnerable to hemodilution, hypothermia, and other variables in surgical settings (Siegel (2002) New England Journal of Medicine, 347:1030-1034). Bleeding may be of particular concern in cardiac surgical patients because large doses of DTIs are used in patients with concurrent organ dysfunction. Renal dysfunction affects metabolism of hirudin and to a lesser degree bivalirudin, and severe hemorrhagic episodes have been reported after cardiopulmonary bypass (Nowak et al. (1992) Thrombosis Research, 66:707-715; Koster et al. (2000) Annals of Thoracic Surgery, 69:37-41; Hein et al. (2005) Artificial Organs, 29:507-510).

In the past, different antidotes for DTIs, particularly for hirudin, have been experimentally researched, including gamma thrombin preparations like DFP thrombin or benzoyl thrombin (Bruggener et al. (1989) Pharmazie, 44:648-9); and the prothrombin intermediate meizothrombin (U.S. Pat. No. 5,817,309; Nowak and Bucha (1995) Thromb. Res., 80:317-25). The chromogenic substrate Chromozym TH (Roche, Mannheim) has been also considered as a potential antidote for melagatran in vitro, although there is no data for its in vivo efficacy (Bodendiek et al. (2000) Hamostaseologie, 23:97-8). To date, such preparations have not been successful in practice because they are too toxic or they are not effective in fluid phase.

Because of the limitations described above, improved methods for the safe and efficient reversal of both heparin and DTIs are needed.

### SUMMARY OF THE INVENTION

Methods for inhibiting the effect of anticoagulants in vivo or in vitro are provided. In particular, a method of inhibiting the anticoagulation effect of a thrombin inhibitor in a patient in need thereof is provided comprising administration of a therapeutically effective amount of a variant prothrombin or thrombin that is capable of binding the thrombin inhibitor and that has reduced procoagulant activity. Variant prothrombins or thrombins of use in the methods of the present invention include thrombin mutants W215A, W215A/E217A, or variants thereof in which the amino acids at positions 215 and/or 217 are alanine. In another embodiment of the present invention, methods are provided in which the thrombin mutants are administered with an additional active agent, particularly hemostatic agents such as activated factor VII or activated prothrombin complex concentrate. In one embodiment of the invention, the methods are useful in the treatment of patients in which a direct thrombin inhibitor has been administered, particularly argatroban.

Methods are also provided for quantifying the concentration of an anticoagulant in the plasma or whole blood of a patient using a variant prothrombin or thrombin titration assay. In particular, the present invention provides a method for quantifying the concentration of a thrombin inhibitor, particularly a DTI, in the plasma or whole blood of a patient. The method comprises dividing a plasma or whole blood sample containing a thrombin inhibitor into testing samples of equivalent volumes that can be separately added to various concentrations of a thrombin mutant and comparing the onset to clotting time for each testing sample. By selecting the testing sample with the shortest onset to clotting time, the concentration of the corresponding thrombin mutant can then be used to estimate the concentration of thrombin inhibitor in the plasma or whole blood sample. Preferred thrombin mutants for use in these methods include W215A/E217A or W215A or variants thereof.

### BRIEF DESCRIPTION OF THE DRAWINGS

FIG. 1 shows the interaction of fibrinogen or thrombin inhibitors with wild-type thrombin or mutant thrombin W215A/E217A (WE). FIG. 1A shows how thrombin's exosite I is necessary for initial fibrinogen binding, while the catalytic site cleaves fibrinogen into fibrin. FIG. 1B shows how bivalirudin and hirudin block both the catalytic site and exosite I of thrombin. FIG. 1C shows how argatroban and FPRck (PPACK) block only thrombin's catalytic site. FIG. 1D shows how two mutations in thrombin's catalytic domain reduces the ability of the resulting mutant thrombin (WE) to cleave fibrinogen and activate platelets. FIG. 1E shows how the mutant thrombin (WE) retains its ability to activate protein C in the presence of thrombomodulin.

FIG. 2 shows a schematic diagram depicting a mechanism by which a thrombin mutant could reverse the effect of an anticoagulant. The thrombin mutant preferentially binds to DTIs, reducing the extent to which endogenous thrombin binds to DTIs and allowing fibrinogen cleavage and activation of platelets. If excess thrombin or thrombin mutant remains in circulation, protein C is activated via interaction with thrombomodulin.

FIG. 3 shows a schematic description of DTI reversal with the thrombin mutant W215A/E217A. Mean values of activated partial thromboplastin time (APTT) are shown in seconds for various DTIs at various concentrations. FIG. 3A shows bivalirudin at 5, 10, and 15 μg/ml. FIG. 3B shows lepirudin at 0.1, 1, and 5 μg/ml. FIG. 3C shows argatroban 0.5 and 1 μg/ml. FIG. 3D shows heparin 0.2 and 0.5 U/ml. The asterisk denotes a statistical difference between samples with and without W215A/E217A at respective anticoagulant levels. APTT values are shortened by W215A/E217A at 5 μg/ml at respective anticoagulant levels. The active-site occupied thrombin (FPR-TH) does not shorten APTTs.

FIG. 4 shows representative Thrombelastogarphy (TEG®) tracings to measure the effect of W215A/E217A on clot formation in the presence of lepirduin or heparin. FIG. 4A shows that Lepirduin prolongs the reaction (lag) time of clot formation (trace 2), and addition of W215A/E217A normalizes clot onset (trace 1). In the presence of soluble thrombomodulin (0.75 μg/ml), W215A/E217A, 5 μg/ml, prolongs clot onset and reduces the clot strength (trace 3). FIG. 4B shows that unfractionated heparin (0.5 U/ml) obliterates clot formation (trace 7) in contrast to the control (trace 4). Protamine sulfate, 5 μg/ml, restores the onset completely, and to a lesser extent clot strength (trace 5). Addition of W215A/E217A, 5 μg/ml, shortens the onset (trace 6), but clot formation remains to be slower than the protamine-treated sample.

FIG. 5 shows representative TEG® tracings to measure the effect of W215A/E217A on clot formation in the presence of bivalirudin, lepirudin, or argatroban. Mean values of reaction time to clotting are measured in minutes. FIG. 5A shows that reaction times are prolonged in the presence of bivalirudin (5 μg/ml), lepirudin (1 μg/ml), or argatroban (0.5 μg/ml). The reaction time values are shortened by W215A/E217A for each respective anticoagulant (*p<0.01). The difference between control (no anticoagulant) and control with W215A/E217A was not statistically different, but the addition of soluble thrombomodulin (0.75 μg/ml) induced significant prolongation of the reaction time (^{#}p<0.01 vs. control). Protamine sulfate reverses heparin (^{§}p<0.01 vs. heparin only). FIG. 5B shows that mean values of maximum amplitude (mm) were reduced with argatroban (*p<0.05) and heparin (^{#}p<0.01), but not with bivalirudin and lepirudin. Addition of W215A/E217A restored the amplitude for all DTIs, but only partially recovered it for heparin (^{§}p<0.05 vs. protamine sulfate). Amplitude was also reduced with addition of W215A/E217A with soluble thrombomodulin (*p<0.05 vs. control).

FIG. 6 shows representative series of thrombin generation curves. FIG. 6A shows that peak thrombin generation in blood plasma was slightly decreased by the addition of W215A/E217A (WE, 5 μg/ml), and modestly decreased by soluble thrombomodulin (rhsTM, 0.75 μg/ml), Addition of both W215A/E217A and thrombomodulin further blunted thrombin generation. FIG. 6B shows that bivalirudin-induced (Bival, 10 μg/ml) prolongation of clotting lag time can be reversed with W215A/E217A (WE, 5 μg/ml). FIG. 6C shows that lepirudin at 1 and 5 μg/ml blunted thrombin formation and addition of W215A/E217A (WE, 5 μg/ml) shortened the onset and restored peak thrombin generation. FIG. 6D shows that argatroban at 0.5 and 1 μg/ml prolonged the onset and reduced peak thrombin generation. Addition of W215A/E217A (WE, 5 μg/ml) shortened onset, but did not reverse the reduced peak level of thrombin formation. FIG. 6E shows that heparin at 0.2 U/ml blunted thrombin generation. Addition of W215A/E217A (WE, 5 μg/ml) shortened clotting onset, but did not reverse the reduced peak level of thrombin formation in contrast to the sample treated with protamine sulfate.

FIG. 7 shows the relationship of onset of clot formation as measured by activated clotting time (ACT) to the relative concentrations of direct thrombin inhibitor (DTI) and W215A/E217A (WE) in a sample of whole blood or plasma. Onset to clotting time is expected to be shortest when the molecular ratio of WE to DTI is close to 1.0. When WE is in excess, WE is expected to bind to thrombomodulin and activate protein C, leading to longer onset to clotting times.

### DETAILED DESCRIPTION OF THE INVENTION

The present invention provides methods for inhibiting the effect of anticoagulants in vivo or in vitro. In particular, the present invention provides a method of inhibiting the anticoagulation effect of a thrombin inhibitor in a patient in need thereof comprising administration of a therapeutically effective amount of a variant prothrombin or thrombin that is capable of binding the thrombin inhibitor and that has reduced procoagulant activity. Variant prothrombins or thrombins of use in the methods of the present invention include thrombin mutants W215A, W215A/E217A, or variants thereof in which the amino acids at positions 215 and/or 217 are alanine. Methods are also provided in which the thrombin mutants are administered with an additional active agent, particularly hemostatic agents such as activated factor VII or activated prothrombin complex concentrate. In one embodiment of the invention, the methods are useful in the treatment of patients in which a direct thrombin inhibitor has been administered, particularly argatroban.

The term “anticoagulant” as used herein refers to any agent or agents capable of preventing or delaying blood clot formation in vitro and/or in vivo. The term “coagulation” as used herein refers to the process of polymerization of fibrin monomers, resulting in the transformation of blood or plasma from a liquid to a gel phase. Coagulation of liquid blood may occur in vitro, intravascularly or at an exposed and injured tissue surface. In vitro blood coagulation results in a gelled blood that maintains the cellular and other blood components in essentially the same relative proportions as found in non-coagulated blood, except for a reduction in fibrinogen content and a corresponding increase in fibrin. By “blood clot” is intended a viscous gel formed of, and containing all, components of blood in the same relative proportions as found in liquid blood.

The phrase “inhibit the anticoagulation effect” as used herein refers to decreasing the ability of an anticoagulant to prevent or delay blood clot formation. Methods for determining whether the anticoagulation effect of an anticoagulant has been inhibited include the use of assays for measuring clot strength and/or the length of time before clot formation in plasma or whole blood samples. Accordingly, inhibiting the anticoagulation effect of an anticoagulant as used herein refers to at least partially reversing the effect of an anticoagulant, including at least 5% reversal, at least 10% reversal, at least 20% reversal, at least 30% reversal, at least 40% reversal, at least 50% reversal, at least 60% reversal, at least 70% reversal, at least 80% reversal, at least 90% reversal, and up to and including 100% reversal. The term “reversal” as used herein refers to a shortening of the time to onset of clot formation or an increase in clot strength. Assays for measuring the onset of clot formation and clot strength are well known in the art and include activated partial thromboplastin time (APTT), thromboelastography (TEG®), and continuous monitoring of thrombin generation using the Thrombinoscope® system (see, for example, the Experimental section below; see also Banez et al. (1980) Am. J. Clin. Pathol., 74:569-574; van den Besselaar et al. (1990) Thromb. Haemost., 63:16-23; Kawasaki et al. (2004) Anesthesia & Analgesia, 99:1440-1444; Hemker et al. (2003) Pathophysiology of Haemostasis & Thrombosis, 33:4-15).

In one embodiment of the invention, methods are provided for the use of a variant prothrombin or thrombin to inhibit the anticoagulation effect of an anticoagulant. Human thrombin is generated from a precursor polypeptide, prothrombin, of approximately 579 mature amino acids (subject to potential allelic variation or N-terminal microheterogeneity) plus a presequence of about 43 residues (Degen et al. (1993) Biochemistry 22:2087). The presequence is proteolytically removed during expression and secretion of prothrombin.

Prothrombin is a zymogen, or inactive protease, that is activated by a series of proteolytic cleavages. At least three sites are subject to cleavage. In vivo, prothrombin is cleaved between residues 8271 and T272 (residue numbers as described in Degen et al. (1993) Biochemistry 22:2087) by Factor Xa in the presence of Factor Va, phospholipid and calcium ions to yield prothrombin 2 and Fragment 1.2. Prothrombin is further proteolytically cleaved by the same system between residues R320 and 1321 to yield meizothrombin, which in turn cleaves autolytically between R155 and 5156 to produce Fragment 1 (1-155) and meizothrombin des 1 (a disulfide linked dipeptide extending from residue 156 to the carboxy terminus of prothrombin, and cleaved at R323). Finally, thrombin is generated from prethrombin 2 by proteolytic cleavage between R320 and 1321, or from meizothrombin des 1 by proteolytic cleavage between R271 and T272. Thrombin itself then autocleaves between T284 and T285 to generate the mature A-chain N-terminus.

The term “thrombin” as used herein refers to a multifunctional prothrombin-derived enzyme. Thrombin acts as a procoagulant by the proteolytic cleavage of fibrinogen to fibrin. It also activates the clotting factors V, VIII, XI and XIII leading to perpetuation of clotting, and the cleavage of the platelet thrombin receptor PAR-1 leading to platelet activation. Multiple antithrombotic mechanisms limit thrombin generation and activity. When thrombin binds to thrombomodulin (TM), an integral membrane protein on vascular endothelial cells, thrombin undergoes a conformational change and loses its procoagulant activity. It then acquires the ability to convert protein C (PC) to activated protein C (APC). APC, a serine protease, acts as a potent anticoagulant by inactivating activated FV (FVa) and FVIII (FVIIIa), two essential cofactors in the clotting or coagulation cascade. APC also inactivates plasminogen activator inhibitor-1 (PAI-1), the major physiologic inhibitor of tissue plasminogen activator (tPA), thus potentiating normal fibrinolysis.

The term “coagulation cascade” as used herein refers to three interconnecting enzyme pathways as described, for example, by Manolin in Wilson et al. (eds): Harrison's Principle of Internal Medicine, 14^{th} Ed. New York. McGraw-Mill, 1998, p. 341, incorporated herein by reference in its entirety. The intrinsic coagulation pathway leads to the formation of Factor IXa, that in conjunction with Factors VIIIa and X, phospholipid and Ca²⁺ gives Factor Xa. The extrinsic pathway gives Factor Xa and IXa after the combination of tissue factor and factor VII. The common coagulation pathway interacts with the blood coagulation Factors V, VIII, IX and X to cleave prothrombin to thrombin (Factor IIa), which is then able to cleave fibrinogen to fibrin.

Two distinct amino acid numbering systems are in use for thrombin in addition to the DNA-based system of Degen et al. (Degen et al. (1993) Biochemistry, 22:2087). One is based on alignment with chymotrypsinogen as described by Bode et al. and is the numbering system used most widely in the protease field (Bode et al. (1989) EMBO. J., 8:3467-3475). In a second, the Sadler numbering scheme, the B chain of thrombin commences with I1 and extends to E259, while the A chain is designated with “a” postscripts, as in T1a to R36a. For example, Wu et al. have disclosed several thrombin mutants numbered in accordance with the Sadler scheme (Wu et al. (1991) Proc. Natl. Acad. Sci. U.S.A., 88:6775-6779). The Wu et al. mutants and the corresponding chymotrypsinogen and Degen et al. residue numbers, respectively, are sequentially shown as follows: H43 (57, 363), K52 (60f, 372), N53 (60 g, 373), R62 (67, 382), R68 (73, 388), R70 (75, 390) D99 (102, 419) and S205 (195, 525).

Throughout the present specification, the Bode et al. numbering system is used to refer to amino acid residues for thrombin and thrombin mutants. However, for the sequence listings corresponding to human thrombin mutant W215A/E217A (SEQ ID NO:1), human thrombin mutant W215A (SEQ ID NO:2), and human thrombin (SEQ ID NO:3), a sequential numbering system is used. Accordingly, amino acid positions 215 and 217 of thrombin and thrombin mutants as described in the present specification using the Bode et al. system correspond to amino acid positions 263 and 265 of thrombin and thrombin mutants in the sequential numbering system used in SEQ ID NOS:1, 2, and 3. A side-by-side comparison of the amino acid sequence for thrombin using the Bode et al. system vs. the sequential numbering system used in SEQ ID NOS:1, 2, and 3 is provided in Table 1. As listed in Table 1, the thrombin A-chain starts at amino acid number 1 of the sequential numbering system, while the thrombin B-chain starts at amino acid number 37.

TABLE 1
Comparison of the Bode et al and Sequential
Numbering Systems for the Amino Acid
Sequence of Human Thrombin

| Sequential No. | Bode et al. No. | Amino Acid |
|---|---|---|
| 1 | 1h | THR |
| 2 | 1g | PHE |
| 3 | 1f | GLY |
| 4 | 1e | SER |
| 5 | 1d | GLY |
| 6 | 1c | GLU |
| 7 | 1b | ALA |
| 8 | 1a | ASP |
| 9 | 1 | CYS |
| 10 | 2 | GLY |
| 11 | 3 | LEU |
| 12 | 4 | ARG |
| 13 | 5 | PRO |
| 14 | 6 | LEU |
| 15 | 7 | PHE |
| 16 | 8 | GLU |
| 17 | 9 | LYS |
| 18 | 10 | LYS |
| 19 | 11 | SER |
| 20 | 12 | LEU |
| 21 | 13 | GLU |
| 22 | 14 | ASP |
| 23 | 14a | LYS |
| 24 | 14b | THR |
| 25 | 14c | GLU |
| 26 | 14d | ARG |
| 27 | 14e | GLU |
| 28 | 14f | LEU |
| 29 | 14g | LEU |
| 30 | 14h | GLU |
| 31 | 14i | SER |
| 32 | 14j | TYR |
| 33 | 14k | ILE |
| 34 | 14l | ASP |
| 35 | 14m | GLY |
| 36 | 15 | ARG |
| 37 | 16 | ILE |
| 38 | 17 | VAL |
| 39 | 18 | GLU |
| 40 | 19 | GLY |
| 41 | 20 | SER |
| 42 | 21 | ASP |
| 43 | 22 | ALA |
| 44 | 23 | GLU |
| 45 | 24 | ILE |
| 46 | 25 | GLY |
| 47 | 26 | MET |
| 48 | 27 | SER |
| 49 | 28 | PRO |
| 50 | 29 | TRP |
| 51 | 30 | GLN |
| 52 | 31 | VAL |
| 53 | 32 | MET |
| 54 | 33 | LEU |
| 55 | 34 | PHE |
| 56 | 35 | ARG |
| 57 | 36 | LYS |
| 58 | 36a | SER |
| 59 | 37 | PRO |
| 60 | 38 | GLN |
| 61 | 39 | GLU |
| 62 | 40 | LEU |
| 63 | 41 | LEU |
| 64 | 42 | CYS |
| 65 | 43 | GLY |
| 66 | 44 | ALA |
| 67 | 45 | SER |
| 68 | 46 | LEU |
| 69 | 47 | ILE |
| 70 | 48 | SER |
| 71 | 49 | ASP |
| 72 | 50 | ARG |
| 73 | 51 | TRP |
| 74 | 52 | VAL |
| 75 | 53 | LEU |
| 76 | 54 | THR |
| 77 | 55 | ALA |
| 78 | 56 | ALA |
| 79 | 57 | HIS |
| 80 | 58 | CYS |
| 81 | 59 | LEU |
| 82 | 60 | LEU |
| 83 | 60a | TYR |
| 84 | 60b | PRO |
| 85 | 60c | PRO |
| 86 | 60d | TRP |
| 87 | 60e | ASP |
| 88 | 60f | LYS |
| 89 | 60g | ASN |
| 90 | 60h | PHE |
| 91 | 60i | THR |
| 92 | 61 | GLU |
| 93 | 62 | ASN |
| 94 | 63 | ASP |
| 95 | 64 | LEU |
| 96 | 65 | LEU |
| 97 | 66 | VAL |
| 98 | 67 | ARG |
| 99 | 68 | ILE |
| 100 | 69 | GLY |
| 101 | 70 | LYS |
| 102 | 71 | HIS |
| 103 | 72 | SER |
| 104 | 73 | ARG |
| 105 | 74 | THR |
| 106 | 75 | ARG |
| 107 | 76 | TYR |
| 108 | 77 | GLU |
| 109 | 77a | ARG |
| 110 | 78 | ASN |
| 111 | 79 | ILE |
| 112 | 80 | GLU |
| 113 | 81 | LYS |
| 114 | 82 | ILE |
| 115 | 83 | SER |
| 116 | 84 | MET |
| 117 | 85 | LEU |
| 118 | 86 | GLU |
| 119 | 87 | LYS |
| 120 | 88 | ILE |
| 121 | 89 | TYR |
| 122 | 90 | ILE |
| 123 | 91 | HIS |
| 124 | 92 | PRO |
| 125 | 93 | ARG |
| 126 | 94 | TYR |
| 127 | 95 | ASN |
| 128 | 96 | TRP |
| 129 | 97 | ARG |
| 130 | 97a | GLU |
| 131 | 98 | ASN |
| 132 | 99 | LEU |
| 133 | 100 | ASP |
| 134 | 101 | ARG |
| 135 | 102 | ASP |
| 136 | 103 | ILE |
| 137 | 104 | ALA |
| 138 | 105 | LEU |
| 139 | 106 | MET |
| 140 | 107 | LYS |
| 141 | 108 | LEU |
| 142 | 109 | LYS |
| 143 | 110 | LYS |
| 144 | 111 | PRO |
| 145 | 112 | VAL |
| 146 | 113 | ALA |
| 147 | 114 | PHE |
| 148 | 115 | SER |
| 149 | 116 | ASP |
| 150 | 117 | TYR |
| 151 | 118 | ILE |
| 152 | 119 | HIS |
| 153 | 120 | PRO |
| 154 | 121 | VAL |
| 155 | 122 | CYS |
| 156 | 123 | LEU |
| 157 | 124 | PRO |
| 158 | 125 | ASP |
| 159 | 126 | ARG |
| 160 | 127 | GLU |
| 161 | 128 | THR |
| 162 | 129 | ALA |
| 163 | 129a | ALA |
| 164 | 129b | SER |
| 165 | 129c | LEU |
| 166 | 130 | LEU |
| 167 | 131 | GLN |
| 168 | 132 | ALA |
| 169 | 133 | GLY |
| 170 | 134 | TYR |
| 171 | 135 | LYS |
| 172 | 136 | GLY |
| 173 | 137 | ARG |
| 174 | 138 | VAL |
| 175 | 139 | THR |
| 176 | 140 | GLY |
| 177 | 141 | TRP |
| 178 | 142 | GLY |
| 179 | 143 | ASN |
| 180 | 144 | LEU |
| 181 | 145 | LYS |
| 182 | 146 | GLU |
| 183 | 147 | THR |
| 184 | 148 | TRP |
| 185 | 149 | THR |
| 186 | 149a | ALA |
| 187 | 149b | ASN |
| 188 | 149c | VAL |
| 189 | 149d | GLY |
| 190 | 149e | LYS |
| 191 | 150 | GLY |
| 192 | 151 | GLN |
| 193 | 152 | PRO |
| 194 | 153 | SER |
| 195 | 154 | VAL |
| 196 | 155 | LEU |
| 197 | 156 | GLN |
| 198 | 157 | VAL |
| 199 | 158 | VAL |
| 200 | 159 | ASN |
| 201 | 160 | LEU |
| 202 | 161 | PRO |
| 203 | 162 | ILE |
| 204 | 163 | VAL |
| 205 | 164 | GLU |
| 206 | 165 | ARG |
| 207 | 166 | PRO |
| 208 | 167 | VAL |
| 209 | 168 | CYS |
| 210 | 169 | LYS |
| 211 | 170 | ASP |
| 212 | 171 | SER |
| 213 | 172 | THR |
| 214 | 173 | ARG |
| 215 | 174 | ILE |
| 216 | 175 | ARG |
| 217 | 176 | ILE |
| 218 | 177 | THR |
| 219 | 178 | ASP |
| 220 | 179 | ASN |
| 221 | 180 | MET |
| 222 | 181 | PHE |
| 223 | 182 | CYS |
| 224 | 183 | ALA |
| 225 | 184 | GLY |
| 226 | 184a | TYR |
| 227 | 185 | LYS |
| 228 | 186 | PRO |
| 229 | 186a | ASP |
| 230 | 186b | GLU |
| 231 | 186c | GLY |
| 232 | 186d | LYS |
| 233 | 187 | ARG |
| 234 | 188 | GLY |
| 235 | 189 | ASP |
| 236 | 190 | ALA |
| 237 | 191 | CYS |
| 238 | 192 | GLU |
| 239 | 193 | GLY |
| 240 | 194 | ASP |
| 241 | 195 | SER |
| 242 | 196 | GLY |
| 243 | 197 | GLY |
| 244 | 198 | PRO |
| 245 | 199 | PHE |
| 246 | 200 | VAL |
| 247 | 201 | MET |
| 248 | 202 | LYS |
| 249 | 203 | SER |
| 250 | 204 | PRO |
| 251 | 204a | PHE |
| 252 | 204b | ASN |
| 253 | 205 | ASN |
| 254 | 206 | ARG |
| 255 | 207 | TRP |
| 256 | 208 | TYR |
| 257 | 209 | GLN |
| 258 | 210 | MET |
| 259 | 211 | GLY |
| 260 | 212 | ILE |
| 261 | 213 | VAL |
| 262 | 214 | SER |
| 263 | 215 | TRP |
| 264 | 216 | GLY |
| 265 | 217 | GLU |
| 266 | 219 | GLY |
| 267 | 220 | CYS |
| 268 | 221 | ASP |
| 269 | 221a | ARG |
| 270 | 222 | ASP |
| 271 | 223 | GLY |
| 272 | 224 | LYS |
| 273 | 225 | TYR |
| 274 | 226 | GLY |
| 275 | 227 | PHE |
| 276 | 228 | TYR |
| 277 | 229 | THR |
| 278 | 230 | HIS |
| 279 | 231 | VAL |
| 280 | 232 | PHE |
| 281 | 233 | ARG |
| 282 | 234 | LEU |
| 283 | 235 | LYS |
| 284 | 236 | LYS |
| 285 | 237 | TRP |
| 286 | 238 | ILE |
| 287 | 239 | GLN |
| 288 | 240 | LYS |
| 289 | 241 | VAL |
| 290 | 242 | ILE |
| 291 | 243 | ASP |
| 292 | 244 | GLN |
| 293 | 245 | PHE |
| 294 | 246 | GLY |
| 295 | 247 | GLU |

The term “variant” or “variant prothrombin or thrombin” as used herein refers to modified amino acid sequences derived from that of prothrombin or thrombin, and which have amino acid substitutions at residue positions 215 and/or 217 of thrombin. Such variants may also be referred to as thrombin mutants. Generally, such variants 45%, 50%, 55%, 60%, 65%, 70%, 75%, 80%, 85%, 90%, 91%, 92%, 93%, 94%, 95%, 96%, 97%, 98%, 99% or more sequence identity to amino acid sequences derived from that of prothrombin or thrombin that have amino acid substitutions at residue positions 215 and/or 217 of thrombin, as determined by sequence alignment programs and parameters described elsewhere herein. Such biologically active variants for use in the methods of the invention may differ from prothrombin or thrombin by as few as 1-15 amino acid residues, as few as 1-10, such as 6-10, as few as 5, as few as 4, 3, 2, or even 1 amino acid residue.

The terms “percent sequence identity” or “percent sequence similarity” as used herein refer to the degree of sequence identity between two sequences as determined using the algorithm of Karlin and Altschul (1990) Proc. Natl. Acad. Sci. USA 87: 2264, modified as in Karlin and Altschul (1993) Proc. Natl. Acad. Sci. USA 90: 5873-5877. Such an algorithm is incorporated into the NBLAST and XBLAST programs of Altschul et al. (1990) J. Mol. Biol. 215: 403-410. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (1997) Nucleic Acids Res. 25: 3389-3402. When utilizing BLAST and Gapped BLAST programs, the default parameters of the respective programs (e.g. XBLAST and NBLAST) are used (See http://www.ncbi.nlm.nih.gov). Other algorithms, equivalent programs, and default settings may also be suitable. By “equivalent program” is intended any sequence comparison program that, for any two sequences in question, generates an alignment having identical amino acid residue matches and an identical percent sequence identity when compared to the corresponding alignment generated by GAP Version 10.

The variant prothrombins or thrombins used in the methods of the present invention are biologically active, that is they possess the desired biological activity of inhibiting the anticoagulation effect of an anticoagulant, as described elsewhere herein. In addition, such variant prothrombins or thrombins exhibit the property of binding to an anticoagulant such as a thrombin inhibitor, particularly a DTI, and typically have reduced procoagulant activity compared to thrombin.

Binding assays for determining the ability of a variant prothrombin or thrombin to bind to anticoagulants such as thrombin inhibitors, including DTIs, are well known in the art (See, for example, the Experimental Section below, as well as Kelly et al. (1992) Proc. Natl. Acad. Sci. USA, 89:6040-6044; Hosokawa et al. (2001) Biochemical Journal. 354:309-313; Schmitz et al. (1991) Europ. J. Biochem., 195:251-256; Okamoto et al. (1981) Biochem. & Biophys. Res. Comm., 101:440-446). In one embodiment of the present invention, the variant prothrombins and thrombins have both an active (catalytic) site and exosite I available for binding to DTIs. The active site cleft of thrombin is bordered by two prominent insertion loops (i.e., the 60-loop and the 148-loop) which control, in part, the interaction of substrates and inhibitors with the active site (Bode et al. (1989) EMBO J., 8:3467-3475; Le Bonniec et al. (1993) J. Biol. Chem., 268:19055-19061; Le Bonniec et al. (1992) J. Biol. Chem., 267:19341-19348). Exosites I and II are electropositive sites in near-opposition on the surface of thrombin known to bind to a number of substrates (Stubbs and Bode (1993) Thromb. Res., 69:1-58; Bode et al. (1992) Protein Sci., 1:26-471). For example, exosite I is known to bind fibrinogen and fibrin I and II (see, for example, Naski et al. (1990)J. Biol. Chem., 265:13484-13489; Naski and Shafer (1991) J. Biol. Chem., 266:13003-13010), while exosite II is known to bind heparin and other glycosaminoglycans (Bode et al. (1992) Protein Sci., 1:26-471; Gan et al. (1994) J. Biol. Chem., 269:1301-1305).

The term “procoagulant” as used herein refers to agents that initiate or accelerate the process of blood coagulation through the transformation of soluble circulating fibrinogen to an insoluble cross-linked, fibrin network. An exemplary procoagulant is native thrombin, or variants thereof, that has a proteolytic activity capable of cleaving fibrinogen to fibrin. In vitro, the procoagulant will ultimately yield a blood clot. In vivo, a procoagulant will ultimately yield a thrombus under pathological conditions. The term “thrombus” as used herein refers to a coagulated intravascular mass formed from the components of blood that results from a pathological condition of an animal or human. Typically the constituents of a thrombus have relative proportions differing from those of the same components in circulating blood. A thrombus is generated in vivo by a dynamic process that comprises cleavage of fibrinogen to fibrin, the activation of platelets and the adherence thereof to the cross-linked fibrin network.

Reduced procoagulant activity, as used herein, may be determined for a variant prothrombin or thrombin through the calculation of its PA/FC ratio (also called “relative anticoagulant potency” or “RAP”) (see, e.g., Di Cera (1998) Trends Cardiovasc. Med., 8:340-350; Dang et al. (1997) Nat. Biotechnol., 15:146-149). The term “PA/FC ratio” as used herein refers to the ratio of the percent of wild-type protein C activation (PA) activity remaining in a variant prothrombin or thrombin relative to the percent of wild-type fibrinogen clotting (FC) activity remaining in the variant prothrombin or thrombin compared to thrombin. A value of PA/FC greater than 1.0 indicates that the variant prothrombin or thrombin has reduced procoagulant fibrinogen cleavage activity relative to the residual anticoagulant activity resulting from protein C activation.

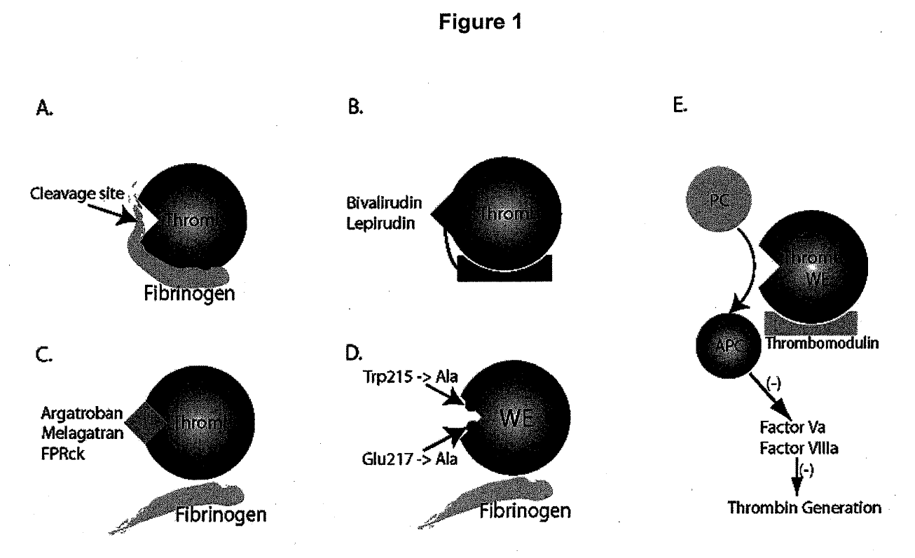

In one embodiment of the present invention, the methods involve use of variant prothrombins or thrombins that include thrombin mutants W215A/E217A (SEQ ID NO:1) and W215A (SEQ ID NO:2), or variants thereof (see U.S. Pat. No. 6,706,512, incorporated herein in its entirety). These thrombin mutants have been previously studied as anticoagulant/antithrombotic agents in vitro and in vivo. As described in more detail in the Experimental Section below, the present invention relates to the finding that these thrombin mutants exhibit the biological activity described above of inhibiting the anticoagulation effect of an anticoagulant. Both W215A/E217A and W215A exhibit substantially reduced fibrinogen cleavage activity compared to thrombin while preserving the capability to activate protein C in the presence of thrombomodulin. When administered intravenously as a sole agent, W215A/E217A and W215A function as an anticoagulant by activating plasma protein C in concert with endothelial thrombomodulin. However, when these thrombin mutants are administered in the presence of thrombin inhibitors such as DTIs they bind directly to the thrombin inhibitors. This binding blocks the inherent anticoagulant activity (via APC activation) of W215A/E217A and W215A. Consequently the proportion of endogenously generated native thrombin bound to DTIs is reduced, thus allowing activation of platelets and fibrinogen (see FIG. 1).

Variants of thrombin mutants W215A/E217A and W215A for use in the methods of the present invention include thrombin mutants sharing at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence of W215A/E217A set forth in SEQ ID NO:1 and the amino acid sequence of W215A set forth in SEQ ID NO:2, and which comprise an alanine residue at the positions corresponding to positions 263 and/or 265 of SEQ ID NOS:1 or 2. As described above, such variants are biologically active (i.e., they inhibit the anticoagulation effect of an anticoagulant), exhibit the property of binding to an anticoagulant such as a thrombin inhibitor, particularly a DTI, and typically have reduced procoagulant activity compared to thrombin.

In another embodiment of the present invention, the methods involve the use of fragments of any of the variant prothrombins or thrombins described herein, so long as such fragments are biologically active (i.e., they inhibit the anticoagulation effect of an anticoagulant), exhibit the property of binding to an anticoagulant such as a thrombin inhibitor, particularly a DTI, and typically have reduced procoagulant activity compared to thrombin. By “fragment” is intended a portion of the amino acid sequence, and generally comprise at least 15, 25, 30, 50, 100, 150, 200, or 250 contiguous amino acids, or up to the total number of amino acids present in a variant prothrombin or thrombin described herein.

In one embodiment of the invention, the methods involve inhibiting the anticoagulation effect of an anticoagulant. Exemplary anticoagulants are thrombin inhibitors, including both indirect thrombin inhibitors and DTIs. Indirect thrombin inhibitors include, for example, heparin, coumarin, dermatan, and thrombomodulin. DTIs include, for example, argatroban or derivatives or analogs thereof, hirudin or recombinant or synthetic derivatives or analogs thereof, derivatives of the tripeptide Phe-Pro-Arg, chloromethylketone derivatives, ximelagatran or derivatives, metabolites, or analogs thereof, an anion binding exosite inhibitor such as Triabin, and an RNA/DNA aptamer (see, e.g., Noeske-Jungblut et al. (1995) J. Biol. Chem., 270:28629-28634; Jeter et al. (2004) FEBS Letters, 568:10-14). Recombinant or synthetic derivatives or analogs (“hirulogs”) of hirudin include, but are not limited to, bivalirudin, lepirudin, and desirudin. Metabolites of ximelagatran include, but are not limited to, melagatran.

In particular embodiments, the DTI is argatroban. Argatroban is a synthetic anticoagulant whose effect is based on the formation of a chemical complex with thrombin's catalytic domain (also called the active site). Binding of argatroban results in a loss of thrombin's catalytic action and in turn results in the inhibition of the activation of platelets and fibrin formation. Argatroban is a small arginine-derived thrombin inhibitor with a molecular weight of 526.66. Owing to its strong affinity for thrombin (Ki values of 0.04 μmol/l) and its direct mechanism of action, its clinical application includes anticoagulation for patients with heparin-induced thrombocytopenia. Despite the beneficial effects, however, clinical use of argatroban has been limited in part because no antidote has been available. Accordingly, in a preferred embodiment of the invention, methods are provided for the inhibition of the anticoagulation effect of argatroban comprising administration to a patient in need thereof a variant prothrombin or thrombin, particularly thrombin mutants W215A/E217A, W215A, and variants thereof.

In another embodiment of the invention, the DTI is the synthetic thrombin inhibitor ximelagatran. Ximelagatran is metabolized in the liver to its active form, melagatran. Analogous to argatroban, melagatran is a catalytic-site directed thrombin inhibitor, and is the first oral form thrombin inhibitor (in contrast with conventional intravenous drugs including argatroban, bivalirudin, and recombinant form of hirudin). Accordingly, in a preferred embodiment of the invention, methods are provided for the inhibition of the anticoagulation effect of ximelegatran and/or its metabolite melagatran comprising administration to a patient in need thereof a variant prothrombin or thrombin, particularly thrombin mutants W215A/E217A, W215A, and variants thereof.

As described above, the present invention provides a method for inhibiting the anticoagulation effect of a thrombin inhibitor in a patient in need thereof comprising administration to the patient of a therapeutically effective amount of thrombin mutants as described herein. By “therapeutically effective amount” is intended an amount of thrombin mutant sufficient to inhibit the anticoagulation effect of an anticoagulant as defined elsewhere herein (i.e., an amount sufficient to at least partially reverse the effect of an anticoagulant, up to and including 100% reversal).

As used herein, the term “patient” refers to any animal, preferably a human, including domestic, agricultural, or exotic animals. In specific embodiments, the human is an adult (over 18 years of age), while in other embodiments, the human is a child (under 18 years of age). The child can be a neonate, infant, toddler, pre-pubescent or post-pubescent and range in age from about birth, 1 month to about 2 year, about 1 year to about 5 years, about 4 years to about 9 years, about 8 years to about 14, or about 13 to about 18 years of age. In addition, the human can be about 55 to 60, 60 to 65, 65 to 70, 70 to 75, 75 to 80, 80 to 85, 85 to 90, 90 to 95 or older.

The thrombin mutants for use in the methods of the present invention can be formulated according to known methods for preparing pharmaceutically useful compositions, such as by admixture with a pharmaceutically acceptable carrier vehicle. Suitable vehicles and their formulation are described, for example, in Remington's Pharmaceutical Sciences (16th ed., Osol, A. (ed.), Mack, Easton Pa. (1980)). In order to form a pharmaceutically acceptable composition suitable for effective administration, such compositions will contain an effective amount of the thrombin mutant, either alone, or with a suitable amount of carrier vehicle.

The term “pharmaceutically acceptable” as used herein refers to a thrombin mutant or other therapeutic agent or compound that while biologically active will not damage the physiology of the recipient human or animal to the extent that the viability of the recipient is comprised.

For use in the methods of the present invention, thrombin mutants as described herein may be administered per se or in the form of a pharmaceutically acceptable salt. When used in medicine, the salts of the thrombin mutant should be both pharmacologically and pharmaceutically acceptable, but non-pharmaceutically acceptable salts may conveniently be used to prepare the free active compound or pharmaceutically acceptable salts thereof and are not excluded from the scope of this invention. Such pharmacologically and pharmaceutically acceptable salts can be prepared by reaction of a thrombin mutant as described herein with an organic or inorganic acid, using standard methods detailed in the literature. Examples of pharmaceutically acceptable salts are organic acids salts formed from a physiologically acceptable anion, such as, tosglate, methenesulfurate, acetate, citrate, malonate, tartarate, succinate, benzoate, etc. Inorganic acid salts can be formed from, for example, hydrochloride, sulfate, nitrate, bicarbonate and carbonate salts. Also, pharmaceutically acceptable salts can be prepared as alkaline metal or alkaline earth salts, such as sodium, potassium, or calcium salts of the carboxylic acid group.

Pharmaceutical compositions may be administered in admixture with a suitable carrier, diluent, or excipient such as sterile water, physiological saline, glucose, or the like. The compositions can contain auxiliary substances such as wetting or emulsifying agents, pH buffering agents, adjuvants, gelling or viscosity enhancing additives, preservatives, flavoring agents, colors, and the like, depending upon the route of administration and the preparation desired.

Pharmaceutical compositions comprising thrombin mutants can be administered in dosages and by techniques well known to those skilled in the medical or veterinary arts, taking into consideration such factors as the age, sex, weight, species and condition of the particular patient, and the route of administration. The route of administration can be via any route that delivers a safe and therapeutically effective dose of a composition of the present invention to the blood of an animal or human. Forms of administration, include, but are not limited to, topical, enteral, and parenteral routes of administration. Enteral routes include oral and gastrointestinal administration. Parenteral routes include intravenous, intraarterial, intramuscular, intraperitoneal, subcutaneous, transdermal, and transmucosal administration. Other routes of administration include epidural or intrathecal administration.

The effective dosage and route of administration are determined by the therapeutic range and nature of the compound, and by known factors, such as the age, weight, and condition of the patient, as well as LD₅₀ and other screening procedures that are known and do not require undue experimentation.

The term “dosage” as used herein refers to the amount of a variant prothrombin or thrombin administered to an animal or human. The therapeutic agent may be delivered to the recipient as a bolus or by a sustained (continuous or intermittent) delivery. When the delivery of a dosage is sustained over a period, which may be in the order of a few minutes to several days, weeks or months, or may be administer chronically for a period of years, the dosage may be expressed as weight of the therapeutic agent/kg body weight of the patient/unit time of delivery.

In one embodiment of the present invention, a variant prothrombin or thrombin is administered as a bolus to a patient in need thereof for the inhibition of the anticoagulation effect of a thrombin inhibitor in a dose of about 0.1 ng to about 500 mg per kg of body weight, about 10 ng to about 300 mg per kg of body weight, from about 100 ng to about 200 mg per kg of body weight, from about 1 μg to about 100 mg per kg of body weight, from about 1 μg to about 50 mg per kg of body weight, or from about 1 μg to about 1 mg per kg of body weight. Alternatively, the amount of variant prothrombin or thrombin administered to achieve a therapeutically effective dose is about 0.1 ng, 1 ng, 10 ng, 100 ng, 200 ng, 300 ng, 400 ng, 500 ng, 600 ng, 700 ng, 800 ng, 900 ng, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 30 μg, 40 μg, 50 μg, 60 μg, 70 μg, 80 μg, 90 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, or 50 mg per kg of body weight or greater. In one aspect of the invention, the variant prothrombin or thrombin is W215A/E217A or W215A or a variant thereof and is administered parenterally, preferably intravenously.

In another embodiment of the present invention, a variant prothrombin or thrombin is administered continuously to a patient in need thereof for the inhibition of the anticoagulation effect of a thrombin inhibitor in a dose of about 0.1 ng, 1 ng, 10 ng, 100 ng, 200 ng, 300 ng, 400 ng, 500 ng, 600 ng, 700 ng, 800 ng, 900 ng, 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 30 μg, 40 μg, 50 μg, 60 μg, 70 μg, 80 μg, 90 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, or 1 mg per kg of body weight per minute or greater. In one aspect of the invention, the variant prothrombin or thrombin is W215A/E217A or W215A or a variant thereof and is administered parenterally, preferably intravenously.

In yet another embodiment of the present invention, a variant prothrombin or thrombin is administered to a patient in need thereof for the inhibition of the anticoagulation effect of a thrombin inhibitor in a dose sufficient to achieve a blood plasma concentration of 0.1 ng/ml, 1 ng/ml, 10 ng/ml, 100 ng/ml, 200 ng/ml, 300 ng/ml, 400 ng/ml, 500 ng/ml, 600 ng/ml, 700 ng/ml, 800 ng/ml, 900 ng/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 30 μg/ml, 40 μg/ml, 50 μg/ml, 60 μg/ml, 70 μg/ml, 80 μg/ml, 90 μg/ml, or 100 μg/ml or greater. In one aspect of the invention, the variant prothrombin or thrombin is W215A/E217A or W215A or a variant thereof and is administered parenterally, preferably intravenously.

In further embodiments of the present invention, combination therapies are provided in which a variant prothrombin or thrombin is the primary active agent and is administered along with an additional active agent to a patient in need thereof for the inhibition of the anticoagulation effect of a thrombin inhibitor. Such combination therapy may be carried out by administration of the different active agents in a single composition, by concurrent administration of the different active agents in different compositions, or by sequential administration of the different active agents. The combination therapy may also include situations where the variant prothrombin or thrombin is already being administered to the patient, and the additional active agent is to be added to the patient's drug regimen, as well as where different individuals (e.g., physicians or other medical professionals) are administering the separate components of the combination to the patient.

The additional active agent will generally, although not necessarily, be one that is effective in inhibiting the anticoagulation effect of a thrombin inhibitor, and/or an agent that enhances or potentiates the effect of the variant prothrombin or thrombin. In a preferred embodiment, the additional active agent is a hemostatic agent, i.e., an agent that promotes hemostasis.

The term “hemostasis” as used herein refers to a coordinated mechanism that maintains the integrity of blood circulation following injury to the vascular system. In normal circulation without vascular injury, platelets are not activated and freely circulate. Vascular injury exposes sub-endothelial tissue to which platelets can adhere. Adherent platelets will attract other circulating platelets to form a preliminary plug that is particularly useful in closing a leak in a capillary or other small vessel. These events are termed primary hemostasis. This is, typically, rapidly followed by secondary hemostasis that involves a cascade of linked enzymatic reactions that result in plasma coagulation to reinforce the primary platelet plug. Accordingly, a hemostatic agent is any agent that slows or stops bleeding by promoting or enhancing any of the physiological processes involved in hemostasis, including contraction of the blood vessels, adhesion and aggregation of formed blood elements, and blood or plasma coagulation.

Particularly preferred hemostatic agents for use in the combination therapies of the present invention include activated factor VII (FVIIa) or activated prothrombin complex concentrate (APCC). Both FVIIa and APCC were developed as hemostatic agents for the treatment of bleeding in patients with inhibitor-developing hemophilia (Scharrer (1999) Haemophilia, 5:253-259; Shapiro et al. (1998) Thromb. Haemost., 80:773-778; Lusher et al. (1980) N Engl. J. Med., 303:421-425; Sjamsoedin et al. (1981) N Engl. J. Med., 305:717-21; Negrier et al. (1997) Thromb. Haemost., 77:1113-1119). The key active ingredient of APCC is prothrombin, which contributes to both hemostasis and thrombus growth (Akhavan et al. (2000) Thromb. Haemost., 84:989-997; Xi et al. (1989) Thromb. Haemost., 62:788-791). By contrast, increasing the plasma concentration of FVIIa is thought to increase the generation of thrombin predominantly through a tissue factor (TF) dependent pathway in which the TF/FVIIa complex activates factors IX and X (Hoffman and Monroe (2001) Thromb. Haemost., 85:958-965).

The present invention also provides methods for quantifying the concentration of an anticoagulant in the plasma or whole blood of a patient using a variant prothrombin or thrombin titration assay. In particular, the present invention provides a method for quantifying the concentration of a thrombin inhibitor, particularly a DTI, in the plasma or whole blood of a patient. As described in more detail in the Experimental Section below, such methods are based on the relationship of the onset of clot formation in a sample of whole blood or plasma to the relative concentrations of thrombin inhibitor and variant prothrombin or thrombin present in the sample. Onset to clotting time is expected to be shortest when the molecular ratio of thrombin inhibitor and variant prothrombin or thrombin is close to 1.0. When the variant prothrombin or thrombin is in excess, these thrombin mutants are expected to bind to thrombomodulin and activate protein C, leading to longer onset to clotting times.

In one embodiment, the method for quantifying the concentration of an anticoagulant involves quantifying the concentration of a thrombin inhibitor, particularly a DTI. The method includes the steps of: a) obtaining a plasma or whole blood sample from the patient; b) adding thrombomodulin to the plasma or whole blood sample; c) loading a series of test chambers with increasing concentrations of a thrombin mutant; d) adding an equivalent amount of the plasma or whole blood sample to each test chamber; e) measuring the onset to clotting time of the plasma or whole blood sample in each test chamber; f) selecting the test chamber with the shortest onset to clotting time; and g) estimating the concentration of the direct thrombin inhibitor in the plasma or whole blood sample as most closely equivalent to the concentration of the thrombin mutant in the selected test chamber as compared to the concentrations of the thrombin mutant in the non-selected test chambers. Preferred thrombin mutants for use in these methods are W215A/E217A or W215A or variants thereof.

With respect to methods for quantifying the concentration of an anticoagulant according to the present invention, onset to clotting time may be measured using any standard test for measuring the onset of clot formation well known in the art, including, for example, APTT as described above or activated clotting time (ACT; see Hattersley (1966) J. Am. Med. Assoc., 196:436-440).

As used herein, the term “test chamber” refers to any apparatus or device in which plasma or whole blood samples may be tested for the onset of clot formation, including, but not limited to, multiwell dishes, single-well dishes, flasks, bottles, or slides.

Having now generally described this invention, the same will be better understood by reference to certain specific examples which are included herein for purposes of illustration only, and are not intended to be limiting of the invention, unless specified.

### Experimental

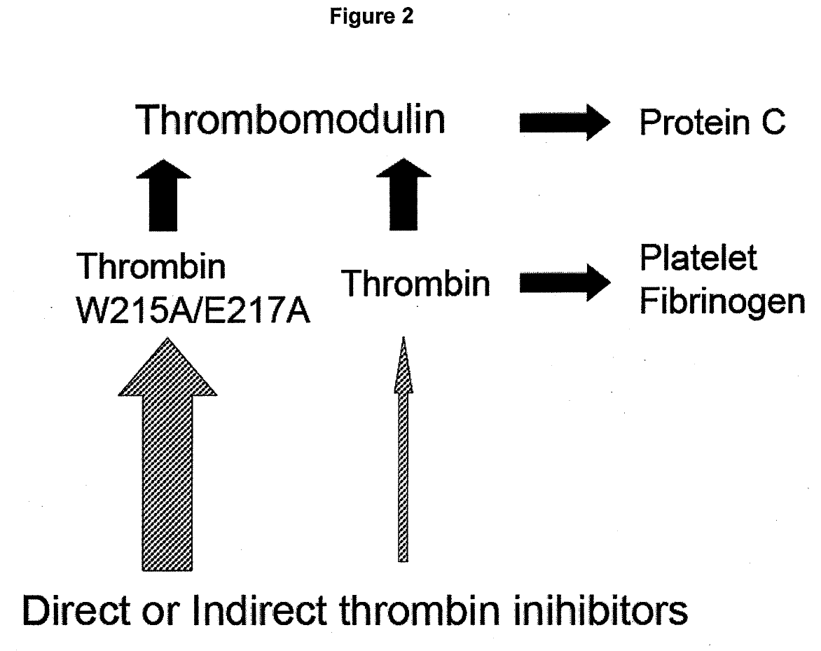

The thrombin mutant, W215A/E217A, has been previously studied as an anticoagulant/antithrombotic agent in vitro and in vivo. The double mutation in its catalytic domain renders W215A/E217A far less potent in platelet activation and fibrinogen cleavage, but preserves capability to activate protein C in presence of thrombomodulin. It was hypothesized that W215A/E217A retains affinity for DTIs, and therefore could be used as a possible reversal agent for these DTIs. In particular, it was hypothesized that, when added to indirect or direct thrombin inhibitors, W215A/E217A first would form a complex with these thrombin inhibitors and reduce the extent of endogenously generated native thrombin bound to thrombin inhibitor, allowing activation of platelets and fibrinogen (see, e.g., FIG. 2).

The effects of W215A/E217A were evaluated in plasma and whole blood samples pretreated with bivalirudin (1-10 μg/ml), lepirudin (0.1-5 μg/ml), argatroban (0.5-1.0 μg/ml), or unfractionated heparin (0.2-0.5 U/ml). The neutralization of DTIs or heparin was evaluated using activated partial thromboplastin time (APTT), thromboelastography (TEG®), and continuous monitoring of thrombin generation (Thrombinoscope®), as described elsewhere herein. For heparin, protamine sulfate was used as standard for comparison.

### EXAMPLE 1

### Use of Thrombin Mutant W215A/E217A to Reverse the Effect of Thrombin Inhibitors

### Materials and Methods

### Materials

The recombinant thrombin mutant, W215A/E217A, was prepared as previously described (Cantwell and Di Cera (2000) Journal of Biological Chemistry, 275:39827-39830). The soluble thrombomodulin was a gift from Asahi Kasei Pharma (Oh-hito, Japan). Tissue factor (TF) was purchased from Dade-Behring (Innovin®, Dade Behring, Marburg, Germany). The direct thrombin inhibitors that were tested include argatroban (GlaxoSmithKline, Research Triangle Park, N.C.), bivalirudin (Angiomax®, Medicines Company, Parsippany, N.J.), and lepirudin (Refludan®, Berlex, Montville, N.J.). The thrombin mutant W215A/E217A was also evaluated against heparin sodium (Elkins-Sinn, Cherry Hill, N.J.), which is antithrombin-dependent indirect inhibitor. Protamine sulfate (Pharmaceutical Partners, East Schaumburg, Ill.) was used as standard for heparin reversal.

Fluorogenic substrate (Z-Gly-Gly-Arg-AMC HCl, molecular weight 616.07) for thrombin generation assay was obtained from Bachem (Switzerland). HEPES (N-2-hydroxyethyl piperazine-N′-2-ethanesulfonic acid), CaCl₂, BSA (bovine serum albumin) and DMSO (dimethyl sulfoxide) were from Sigma (Sigma, St Louis, Mo.). Tissue factor was dissolved in 10 ml sterile water and then further diluted with working HEPES buffer (20 mM HEPES, 140 mM NaCl, 5 mg/ml BSA), (1:75). For the preparation of Fluca-buffer 1.75 ml HEPES buffer (pH 7.35, 20 mM HEPES, 60 mg/ml BSA was added to 0.2 ml of 1 M CaCl₂ in a glass test tube, mixed, and warmed up for few minutes at 37° C. Just before use 50 μl of 100 mM fluorogenic substrate made in DMSO was added to the HEPES-CaCl₂ solution and mixed to dissolve. This buffer now contains 2.5 mM substrate and 100 mM CaCl₂.

For plasma collection, whole blood samples (5 ml, in 3.2% trisodium citrate) were obtained after institutional approval and informed written consent from 6 volunteers that had not received any medication in the preceding 2 weeks. For APTT and thrombin generation experiments blood was centrifuged to obtain platelet poor plasma (PPP) for 20 min at 3000×g. PPP was used immediately or stored for not more than a couple of days at −20° C. For TEG® measurements, whole blood in citrate was used after recalcification within 5 min from collection. In all experiments, the final concentration of recombinant thrombin mutant was 5 μg/ml.

### The Measurement of Activated Partial Thromboplastin Time (APTT)

The efficacy of thrombin mutant was also evaluated in conventional APTT assay using the PTT Automate® on STart® 4 instruments (Diagnostica Stago, Asnieres, France). For measurements of APTT (in seconds), fifty microliter aliquots of platelet-poor plasma were transferred to disposable cuvettes (Diagnostica Stago, Parsippany, N.J.), and after addition of the APTT reagent and pre-incubation at 37° C. samples were run in duplicate. Therapeutic concentrations of DTIs (argatroban 0.5-1 μg/ml; lepirudin 0.1-1 μg/ml; bivalirudin 1-10 μg/ml), and heparin (0.2-0.5 U/ml) were used to pre-treat plasma. Effects of thrombin mutant on APTT in DTI-treated plasma were evaluated after adding thrombin mutant (5 μg/ml, final concentration). Catalytic-site blocked thrombin (thrombin saturated with Phe-Pro-Arg-chlormethylketone; FPRck) was used at 100 μg/ml in some experiments for comparison.

### Thrombelastogarphy (TEG®)

The viscoelastic measurement of clot formation was evaluated using Thrombelastogarphy (TEG®, Haemoscope, Niles, Ill.). The whole blood samples (1 ml, in 3.2% trisodium citrate) were spiked with one of DTIs (bivalirudin 5 μg/ml, lepirudin 1 μg/ml, or argatroban 0.5 μg/ml) or heparin 0.5 U/ml. For TEG® measurements, 360 μl aliquots of the samples were transferred to disposable cups containing 10 of 0.4 M CaCl₂. Effects of W215A/E217A on TEG® variables were evaluated after adding this thrombin mutant (5 μg/ml, final concentration) to respective samples. The TEG® variables, the reaction time (in minutes) which represents the onset of clot formation, and maximum amplitude (in mm) which reflects the strength of clot were compared for different groups (Kawasaki et al. (2004) Anesthesia & Analgesia, 99:1440-1444).

### The Monitoring of Thrombin Generation by Thrombinoscope®

In order to evaluate this complex regulation of thrombin generation, the Thrombinoscope® system, which enables automated acquisition of thrombin generation time courses in platelet-rich plasma or in platelet-poor plasma (Hemker et al. (2003) Pathophysiology of Haemostasis & Thrombosis, 33:4-15). The method for the automated estimation of endogenous thrombin potential using a commercially available fluorogenic substrate (Z-GlyGly-Arg-AMC) has been described in detail elsewhere (Hemker et al. (2003) Pathophysiology of Haemostasis & Thrombosis, 33:4-15). Briefly, for the thrombin generation experiments, 80 μl of PPP, and 20 μl of the thrombin generation trigger are added to wells of 96-well microtiter plate (Microfluor2, Labsystems, Finland), followed by 20 μl of substrate-calcium chloride buffer. The reaction is monitored using microplate fluorometer (Fluoroskan Ascent, Labsystems, Finland) set at 390 nm (excitation wavelength) and 460 nm (emission wavelength). Fluorescence is recorded every 20 sec for 90 minutes and the acquired data are automatically processed by a commercially available Thrombinoscope software (Synapse B.V) that displays the progress of reaction and calculates the thrombin generation parameter (peak thrombin level). The efficacy of the thrombin mutant was tested in PPP pretreated with therapeutic concentrations of DTIs (argatroban 0.5-1 μg/ml; lepirudin 0.1-5 μg/ml; bivalirudin 1-10 μg/ml) in the presence or absence of the thrombin mutant (5 μg/ml). Additionally, heparin (0.2 or 0.5 U/ml) was also evaluated. Heparin reversal with an appropriate concentration of protamine was used for comparison (1 mg of protamine for each 100 units (1 mg) of heparin). To evaluate the effects of the thrombin mutant on protein C activation (Regnault et al. (2003) Thrombosis and Haemostasis, 89:208-212), soluble thrombomodulin (0.75 μg/ml, final concentration) was added to the sample plasma containing the thrombin mutant.

### Data Analysis

The respective experiment was conducted at least in three different individual samples. The tracings for thrombin generation (Thrombinoscope®) and TEG® are representative single sets among three measurements. For statistical analyses, a paired t test (two-tailed) was used to determine the difference between measured variables with and without W215A/E217A.

### Results

### Effects of W215A/E217A on APTT

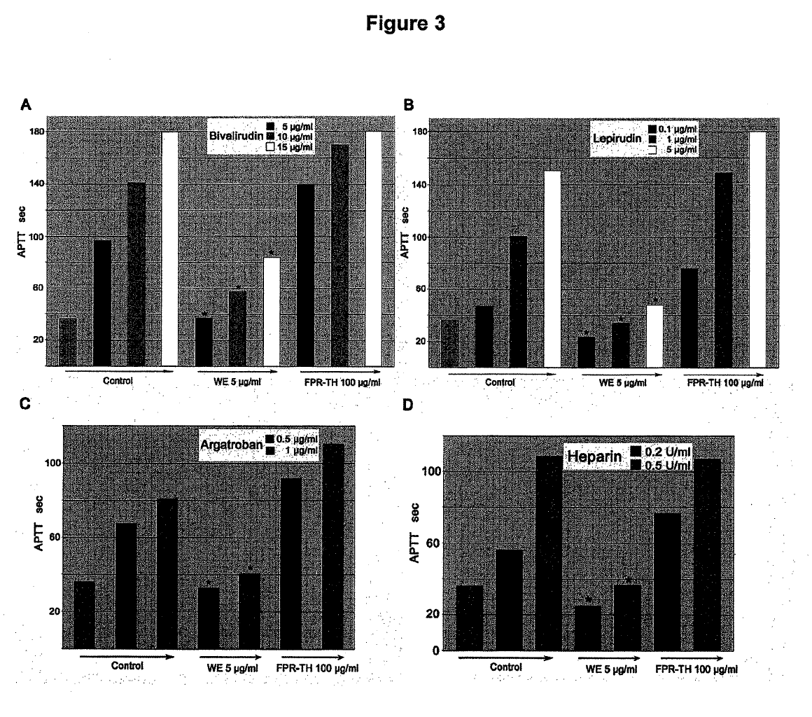

All DTIs and heparin prolong APTT in a concentration dependent manner (FIG. 3, Panels A-D). The addition of W215A/E217A to plasma shifted APTT values toward normal ranges. The active site blocked thrombin (FPR-TH) had no effect on APTT prolonged with either DTIs or heparin.

### Effects of W215A/E217A on TEG®

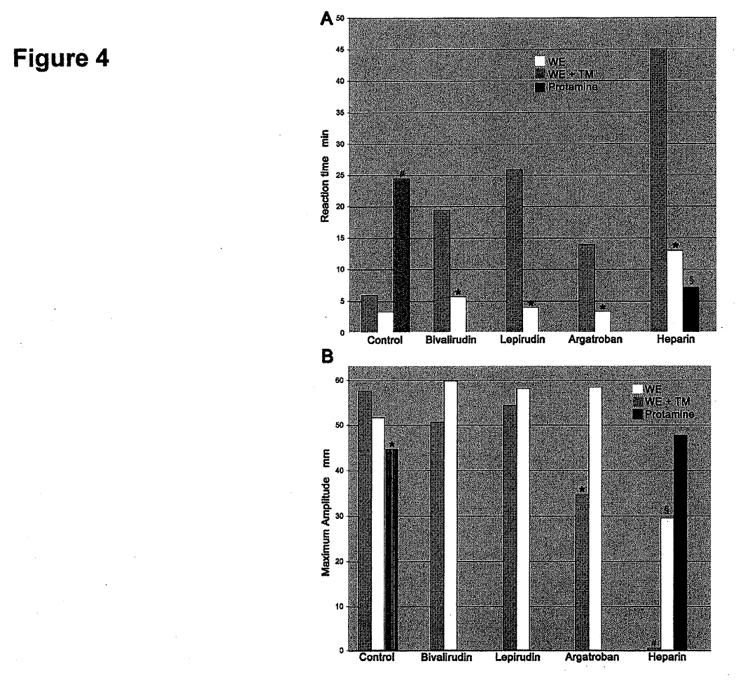

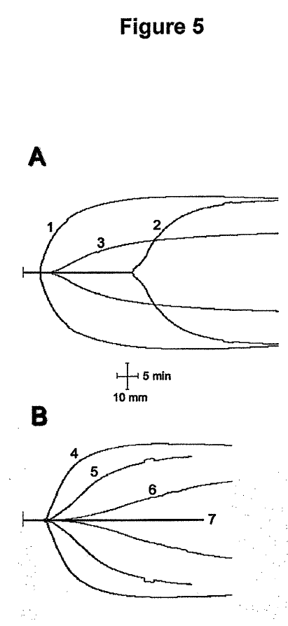

All studied agents prolonged the reaction time by at least twice over the control value (5.9 min) (FIG. 4A). Heparin essentially obliterated clot formation during 45-min period. The strength of clot shown in the amplitude (mm) was minimally affected by lepirudin and bivalirudin, but was reduced by argatroban (FIG. 4B). With addition of W215A/E217A (5 μg/ml), both reaction time and amplitude variables were reversed to normal for all DTIs (FIGS. 4A, 5A). For heparin, the reaction time was notably shortened by W215A/E217A (from 45 min to 14 min, p<0.01), but the amplitude showed modest (incomplete) recovery when compared to protamine sulfate (FIG. 5B). In the presence of soluble thrombomodulin (0.75 μg/ml), W215A/E217A, 5 μg/ml, prolongs the onset and reduces the clot strength (FIG. 5A).

### Effects of W215A/E217A on Thrombin Generation

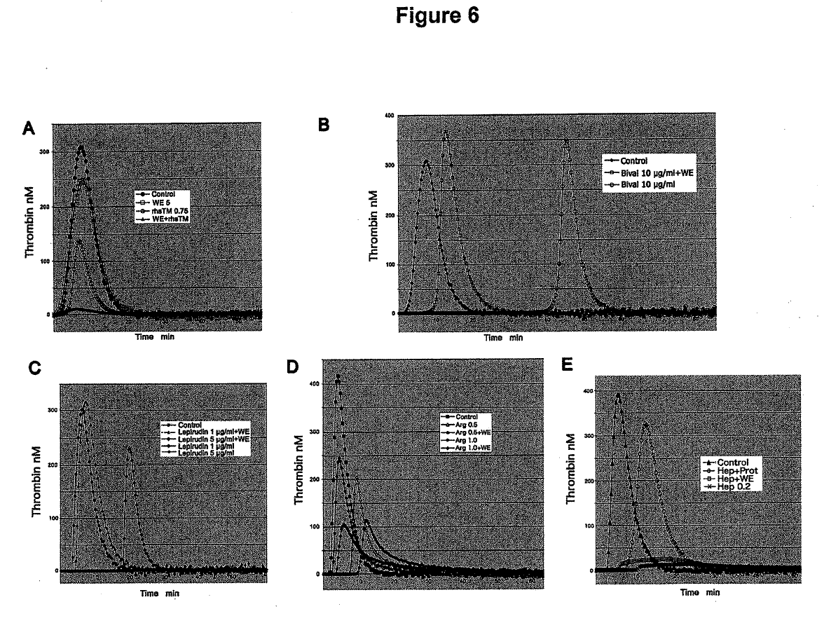

The addition of W215A/E217A, 5 μg/ml, to plasma slightly decreased the peak of thrombin generation, and this effect was substantially potentiated by added soluble thrombomodulin (0.75 μg/ml) (FIG. 6A). Bivalirudin and lepirudin prolonged the onset of thrombin generation, but not the peak level (FIGS. 6B and 6C, respectively). W215A/E217A was effective in shortening prolonged latency of thrombin generation. The addition of argatroban to plasma prolonged the onset and reduced the peak of thrombin generation in a dose-dependent manner (FIG. 6D). The addition of W215A/E217A shortened the onset, but not the peak thrombin generation, although moderate amount (>100 nM) of peak thrombin levels were observed with argatroban at 0.5 and 1 μg/ml (FIG. 6D). In the presence of heparin (0.2 U/ml), thrombin generation was obtunded, and addition of either W215A/E217A, 5 μg/ml, or protamine sulfate, 2 μg/ml, shortened the onset, but only protamine sulfate reversed the peak level of thrombin formation.

### SUMMARY

All antithrombin agents studied caused prolongation of APTT (46.6-180 sec vs. control 36 sec) in a concentration dependent manner, which was reversed with W215A/E217A at 5 μg/ml. DTIs and heparin prolonged the onset of clot formation (>14 min vs. control 5.9 min) and decreased clot strength on TEG®. The thrombin mutant W215A/E217A shortened the lagtime of APTT, thrombin generation, and TEG® that were prolonged by heparin and DTIs. The amount of thrombin generation and subsequent clot formation were fully restored by W215A/E217A in DTI-anticoagulated samples, and was partially recovered in heparin-treated samples. W215A/E217A, 5 μg/ml, effectively reversed TEG® variables in the presence of DTIs, but was modestly effective for heparin in comparison to protamine. On the thrombin generation assay, W215A/E217A, 5 μg/ml, recovered lag time and peak thrombin for both bivalirudin and lepirudin. For argatroban, W215A/E217A notably shortened the lagtime, but not peak thrombin. Although protamine sulfate may have been more efficient in neutralizing heparin, shorter lagtime was observed with W215A/E217A. These results indicate that W215A/E217A can be therapeutically used as a reversal agent for DTIs, and as a possible second line therapy for reversal of heparin anticoagulation.

Thrombin plays important physiological roles in activating platelets and fibrinogen for hemostasis as well as anticoagulant protein C. W215A/E217A is a novel thrombin mutant which exerts protein C activation in the presence of endothelial thrombomodulin, has notably reduce catalytic activity toward platelet PAR-1, fibrinogen and antithrombin (Cantwell and Di Cera (2000) Journal of Biological Chemistry, 275:39827-39830; Gruber et al. (2002) Journal of Biological Chemistry, 277:27581-27584). When administered intravenously as a sole agent, W215A/E217A functions as an anticoagulant activating plasma protein C in concert with endothelial thrombomodulin. However, when administered in the presence of DTIs, W215A/E217A binds directly to thrombin inhibitors, and the inherent anticoagulant activity (APC activation) of W215A/E217A is blocked by DTIs (Linder et al. (1999) Thrombosis Research, 95:117-125). Consequently the proportion of endogenously generated native thrombin bound to DTIs is reduced at the site of injury, thus allowing activation of platelets and fibrinogen (see FIG. 2).

The available data on W215A/E217A strongly supports the binding of bivalirudin whose amino-terminus is FPRP, and carboxyl-terminus is similar to hirudin dodecapeptide (Kelly et al. (1992) Proc. Natl. Acad. Sci. USA, 89:6040-6044). The mechanism of action of lepirudin and bivalirudin is bivalent attachment of thrombin inhibitor to the catalytic domain and exosite I of thrombin. Studies of the crystal structure of FPRck-bound W215A/E217A support binding of the amino-terminus of bivalirudin, and unmodulated exosite I is available for binding of carboxyl-terminal dodecapeptide. The availability of both catalytic and exosite I of thrombin mutant seems important for its DTI reversal because FPRck-bound native thrombin did not show any efficacy, and it has inherent anticoagulant effects (Hosokawa et al. (2001) Biochemical Journal, 354:309-313). This finding underlies the improved binding of bivalent DTIs at two distinct thrombin sites (Schmitz et al. (1991) European Journal of Biochemistry, 195:251-256) because either amino-terminal tripeptide FPR nor carboxyl-terminal dodecapeptide was as efficient antithrombotic as bivalirudin (Kelly et al. (1992) Proc. Natl. Acad. Sci. USA, 89:6040-6044).

In contrast, small molecular thrombin inhibitors such as argatroban and melagatran (the active form of ximelagatran) bind only to thrombin's active site (Okamoto et al. (1981) Biochemical & Biophysical Research Communications, 101:440-446). The present data on argatroban suggests that the catalytic domain of W215A/E217A is accessible to univalent inhibitors in a similar manner as to FPRck.

Heparin mediates thrombin inhibition by antithrombin (AT), an endogenous SERPIN, which is uniquely different from DTIs. This glycosaminoglycan induces conformational changes in AT (Johnson and Huntington (2003) Biochemistry, 42:8712-8719; O'Keeffe et al. (2004) Journal of Biological Chemistry, 279:50267-50273), and the catalytic site of thrombin is irreversibly inhibited by the reactive center loop of AT. These inhibitory reactions are supported by heparin binding to thrombin exosite II. Such exosite interactions between W215A/E217A, and heparin might have occurred because exosites are not modulated in this mutant. However, in contrast to the interaction with DTIs, W215A/E217A minimally increased the peak thrombin generation and partially restored clot strength on TEG® in the presence of AT-heparin. The recovery of APTT with W215A/E217A in the presence of heparin was striking, however the endpoint of APTT is the initial phase of thrombin generation (5-10 nM), and therefore the total amount of thrombin generation is not reflected (Rand et al. (1996) Blood, 88:3432-3445). The affinity of W215A/E217A to AT is severely compromised (Cantwell and Di Cera (2000) Journal of Biological Chemistry, 275:39827-39830), and other thrombin mutants (e.g., E192Q, desETW) also exhibit a loss of affinity to AT which is restored by heparin (Le Bonniec et al. (1995) Biochemistry, 34:12241-12248). The current data showed that addition of W215A/E217A to heparinized blood caused only a small increase in endogenous thrombin generation. This makes W215A/E217A a highly specific DTI reversal agent, and most likely increases its in vivo safety because adsorption of AT-heparin complex may actually lead to AT deficiency and prothrombotic condition (Petaj a et al. (1996) Journal of Thoracic & Cardiovascular Surgery, 112:665-671).

Currently, heparin remains to be the mainstay anticoagulant in cardiac surgery. Despite the rapid reversal of anticoagulation with protamine, severe post-operative bleeding occurs in 3-5% of patients undergoing cardiac surgery with the cardiopulmonary bypass (Dacey et al. (1998) Archives of Surgery, 133:442-447). Patients allergic to protamine may suffer from serious adverse events, and there is no alternative heparin-reversal protocol (Williams et al. (1994) Journal of Thoracic & Cardiovascular Surgery, 108:975-983; Stafford-Smith et al. (2005) Anesthesiology, 103:229-240). If, as demonstrated by the present data, DTIs can be reversed at conclusion of surgery, these agents may be used for either heparin or protamine contra-indication.

The post-cardiac procedural bleeding associated with DTIs are often difficult to control because large dose of potent DTIs are used with concomitant use of potent anti-platelet agents (Maroo and Lincoff (2004) Seminars in Thrombosis & Hemostasis, 30:329-336) or in the presence of thrombocytopenia (Dyke et al. (2005) Annals of Thoracic Surgery, 80:299-303). In a recent clinical trial of bivalirudin and heparin-protamine in off-pump coronary surgery, both groups had comparable 12-hour post-operative chest tube drainage (median, 793 vs. 805 ml), but the range of blood loss widely varied in the bivalirudin group (320-4,909 ml, n=50) (Merry et al. (2004) Annals of Thoracic Surgery, 77:925-931). Unintentional overdosing, hepatic disorders (for argatroban) (Swan and Hursting (2000) Pharmacotherapy, 20:318-329), and renal disorders (for bivalirudin and lepirudin) (Koster et al. (2000) Annals of Thoracic Surgery, 69:37-41; Robson et al. (2002) Clinical Pharmacology & Therapeutics, 71:433-439; Chew et al. (2005) American Journal of Cardiology, 95:581-585) may all contribute to increased risk of bleeding. The necessity of a novel antidote for DTIs is thus obvious.

Different antidote principles, particularly for lepirudin, have been explored to reduce bleeding risks. The use of γ-thrombin preparations, such as diisopropylphosphorothrombin (DIP thrombin) or benzoyl-thrombin (Bruggener et al. (1989) Pharmazie, 4:648-649) and the use of meizothrombin, a prothrombin intermediate (Nowak and Bucha (1995) Thrombosis Research, 80:317-325), have been proposed as a neutralizing agent for hirudin and its synthetic analogues. To date, these preparations have not been successful in practice because they are too toxic or they are not effective in fluid phase. Alternative approaches to DTI-associated bleeding are transfusion of hemostatic factor products including recombinant activated factor VII (NovoSeven®) (Hein et al. (2005) Artificial Organs, 29:507-510; Malherbe et al. (2004) Anesthesiology, 100:443-445) and FEIBA or autoplex preparations (Irani et al. (1995) American Journal of Cardiology, 75:422-423; Elg et al. (2001) Thrombosis Research, 101:159-170), but activated plasma products may be associated with prothrombotic complications (Bui et al. (2002) Journal of Thoracic & Cardiovascular Surgery, 124:852-854; Aledort (2004) Journal of Thrombosis & Haemostasis, 2:1700-1708). It is thus rational for antidotes to be non-thrombogenic in nature. The failure of heparinase Ito replace protamine may be due to the generation of low molecular heparin as well as its potential for degrading endothelial glycosaminoglycans. Conversely, the thrombin mutant, W215A/E217A, increases safety margins because intravenous administration of W215A/E217A results in systemic activation of protein C. This is a corollary to the relative safety of rapid reversal of heparin with protamine, which exerts inherent anticoagulant activity (Chu et al. (2001) British Journal of Haematology, 115:392-399) and potentiation of protein C activation (Slungaard and Key (1994) Journal of Biological Chemistry, 269:25549-25556).

The concentrations of heparin and DTIs used in the current experiments are within therapeutic ranges of heparin (0.5 U/ml; 3.3 mM), argatroban (0.5-1.0 μg/ml; 1-2 μM), bivalirudin (5-10 μg/ml; 2.3-4.6 μM), and lepirudin (1 μg/ml; 0.14 μM). The molar concentration for W215A/E217A, 5 μg/ml or 0.14 μM, was mostly below those of thrombin inhibitors (assuming W215A/E217A as 37 kDa). In addition to the molar ratio, relative difference in Ki values (lepirudin>>bivalirudin>argatroban) toward thrombin may also affect binding to W215A/E217A. It is possible that W215A/E217A restores hemostasis in vivo because of faster onset and sufficient thrombin generation (peak thrombin over 100 nM, FIG. 6D). The in vivo plasma concentration of W215A/E217A may reach 3.75 μg/ml in the non-human primate model (Gruber et al. (2002) Journal of Biological Chemistry, 277:27581-27584), and higher dosing schemes can be safely used in the presence of anticoagulation with heparin and DTIs.

In summary, the present findings demonstrate that variant thrombins and prothrombins with substantially reduced procoagulant activity can be used to recover endogenous thrombin function. Notably, the thrombin mutant W215A/E217A, is available in suitable form for intravenous injection (Gruber et al. (2002) Journal of Biological Chemistry, 277:27581-27584), and additional in vivo studies are underway to confirm its clinical utility.

### EXAMPLE 2

### Titration Assay for Quantitation of DTIs in Plasma or Whole Blood of a Patient

An assay for the quantitation of DTIs in plasma or whole blood of a patient using the thrombin mutant W215A/E217A (WE) is prepared as follows. Predetermined levels of WE are pre-inserted in 6-channel wells as described in Table 2 using a cartridge format based on the Hepcon HMS Plus 6-channel ACT system (Medtronic Perfusion Systems, Minneapolis, Minn.).

TABLE 2
Sample concentrations of WE for use in DTI titration assay

| | Ch1 | Ch2 | Ch3 | Ch4 | Ch5 | Ch6 |
|---|---|---|---|---|---|---|
| WE per ml | 16 μg | 8 μg | 6 μg | 4 μg | 2 μg | 0 μg |
| M.W. 37,000 | 0.44 μM | 0.22 μM | 0.16 μM | 0.11 μM | 0.05 μM | 0 μM |

M.W. = molecular weight

Plasma or whole blood samples containing DTI are taken from a patient. These samples are drawn in a syringe that contains thrombomodulin (0.03 μg/ml, final concentration), an endogenous inhibitors of thrombin. Equivalent amounts of the plasma or whole blood sample are added to each well. Onset to clotting time is measured for the samples in each well using APTT as described above, or activated clotting time (ACT) using methods well known to those of skill in the art (see, for example, Hattersley (1966) J. Am. Med. Assoc., 196:436-440).

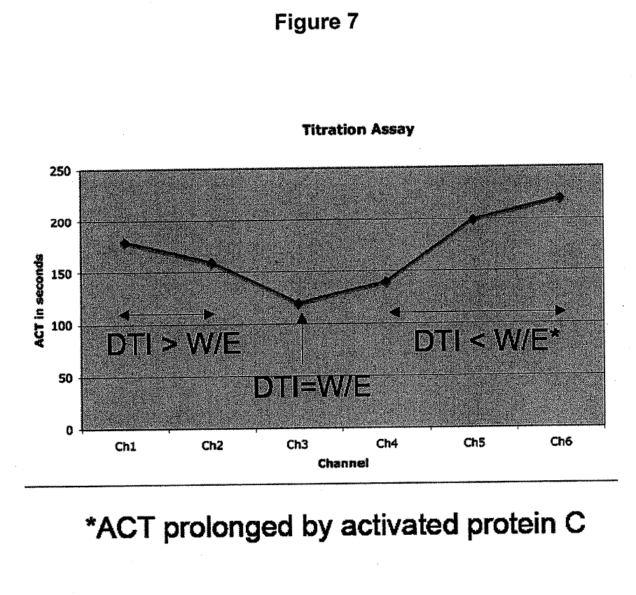

Because DTIs bind to thrombin (or WE) much more quickly than endogenous inhibitors of thrombin such as antithrombin or thrombomodulin (see Table 3). In Table 2, kₒₙ is the association constant for α-thrombin and inhibitor obtained from binding studies in the presteady state phase with stopped flow spectrofluorometry, while kᵢ is the inhibitory constant. The larger the kₒₙ, the more rapid the binding of inhibitor to thrombin. The lower the kᵢ, the more selective and tight the inhibition of thrombin. (See, e.g., Elg et al. (1997) Thrombosis Haemostasis, 78:1286-1292; Aritomi et al. (1993) Thrombosis Haemostasis, 70:418-422). Onset to clotting time is expected to be shortest when the molecular ratio of WE to DTI is close to 1.0 (see FIG. 7). When WE is in excess, WE is expected to bind to thrombomodulin and activate protein C, leading to longer onset to clotting times. Conversely, when more DTI is available, all the WE present is saturated with DTI and although protein C activation is blocked, the excess DTI leads to longer onset to clotting times.

TABLE 3
The rate of binding to WE and inhibitory
constant of thrombin inhibitors

| | M.W. | kₒₙ | kᵢ |
|---|---|---|---|
| | Da | 10⁶ M⁻¹s⁻¹ | ×10⁹ M |
| Argatroban | 582 | 33 | 10 |
| Melagatran | 430 | 12 | 2-4.1 |
| Bivalirudin | 2178 | 430 | 1.9 |
| Lepirudin | 6800 | 290 | 0.00006 |
| Thrombomodulin | 52,000 | 0.053 | 26 |
| Antithrombin | 65,000 | 0.005 | <0.2 |

M.W. = molecular weight

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method of inhibiting the anticoagulation effect of a thrombin inhibitor in a patient in need thereof comprising administering to said patient a therapeutically effective amount of a thrombin mutant, wherein said thrombin mutant comprises an amino acid sequence selected from the group consisting of:
a) the amino acid sequence of thrombin mutant W215A/E217A set forth in SEQ ID NO:1;
b) an amino acid sequence having at least 80% sequence identity to the amino acid sequence set forth in SEQ ID NO:1 and which comprises an alanine residue at the positions corresponding to positions 263 and 265 of SEQ ID NO:1, wherein said amino acid sequence encodes a thrombin mutant that binds to said thrombin inhibitor;
c) an amino acid sequence having at least 90% sequence identity to the amino acid sequence set forth in SEQ ID NO:1 and which comprises an alanine residue at the positions corresponding to positions 263 and 265 of SEQ ID NO:1, wherein said amino acid sequence encodes a thrombin mutant that binds to said thrombin inhibitor;
d) the amino acid sequence of thrombin mutant W215A set forth in SEQ ID NO:2;
e) an amino acid sequence having at least 80% sequence identity to the amino acid sequence set forth in SEQ ID NO:2 and which comprises an alanine residue at the position corresponding to position 263 of SEQ ID NO:2, wherein said amino acid sequence encodes a thrombin mutant that binds to said thrombin inhibitor; and
f) an amino acid sequence having at least 90% sequence identity to the amino acid sequence set forth in SEQ ID NO:2 and which comprises an alanine residue at the position corresponding to position 263 of SEQ ID NO:2, wherein said amino acid sequence encodes a thrombin mutant that binds to said thrombin inhibitor.

2. The method of claim 1, wherein said thrombin inhibitor is a direct thrombin inhibitor.

3. The method of claim 2, wherein said direct thrombin inhibitor is selected from the group consisting of argatroban or derivatives or analogs thereof, hirudin or recombinant or synthetic derivatives or analogs thereof, a derivative of the tripeptide Phe-Pro-Arg, a chloromethylketone derivative, ximelagatran or derivatives, metabolites, or analogs thereof, an anion binding exosite inhibitor, and an RNA/DNA aptamer.

4. The method of claim 3, wherein said direct thrombin inhibitor is argatroban.

5. The method of claim 3, wherein said recombinant or synthetic derivative or analog of hirudin is selected from the group consisting of bivalirudin, lepirudin, and desirudin.

6. The method of claim 3, wherein said metabolite of ximelagatran is melagatran.

7. The method of claim 1, wherein said thrombin inhibitor is an indirect thrombin inhibitor.

8. The method of claim 7, wherein said indirect thrombin inhibitor is heparin, coumarin, dermatan, or thrombomodulin.

9. The method of claim 1, wherein said thrombin mutant comprises the amino acid sequence set forth in SEQ ID NO:1.

10. The method of claim 1, wherein said thrombin mutant comprises the amino acid sequence set forth in SEQ ID NO:2.

11. The method of claim 1, wherein said thrombin mutant is administered to said patient parenterally.

12. The method of claim 11, wherein said thrombin mutant is administered to said patient intraperitoneally, intravenously, subcutaneously, or intramuscularly.

13. The method of claim 12, wherein said thrombin mutant is administered to said patient intravenously.

14. The method of claim 13, wherein said thrombin mutant is administered to said patient in a dose of about 0.1 ng to about 500 mg per kg of body weight.

15. The method of claim 13, wherein said thrombin mutant is administered in to said patient in a dose sufficient to achieve a blood plasma concentration of at least 3 μg/ml.

16. The method of claim 15, wherein said thrombin mutant is administered in to said patient in a dose sufficient to achieve a blood plasma concentration of 3.75 μg/ml.

17. The method of claim 1, further comprising administration of a hemostatic agent to said patient.

18. The method of claim 17, wherein said hemostatic agent is activated factor VII or activated prothrombin complex concentrate.

19. The method of claim 17, wherein said thrombin mutant and said hemostatic agent are administered concurrently.

20. The method of claim 17, wherein said thrombin mutant and said hemostatic agent are administered sequentially.

21. A method for the quantitation of the concentration of a direct thrombin inhibitor in the plasma or whole blood of a patient, comprising the steps of:
a) obtaining a plasma or whole blood sample from said patient;
b) adding thrombomodulin to said plasma or whole blood sample;
c) loading a series of test chambers with increasing concentrations of a thrombin mutant;
d) adding an equivalent amount of said plasma or whole blood sample to each test chamber;
e) measuring the onset to clotting time of said plasma or whole blood sample in each test chamber;
f) selecting the test chamber with the shortest onset to clotting time; and
g) estimating the concentration of said direct thrombin inhibitor in said plasma or whole blood sample as most closely equivalent to the concentration of said thrombin mutant in the selected test chamber as compared to the concentrations of said thrombin mutant in the non-selected test chambers.

22. The method of claim 21, wherein said thrombin mutant comprises an amino acid sequence selected from the group consisting of:
a) the amino acid sequence of thrombin mutant W215A/E217A set forth in SEQ ID NO:1;
b) an amino acid sequence having at least 80% sequence identity to the amino acid sequence set forth in SEQ ID NO:1 and which comprises an alanine residue at the positions corresponding to positions 263 and 265 of SEQ ID NO:1, wherein said amino acid sequence encodes a thrombin mutant that binds to said thrombin inhibitor;
c) an amino acid sequence having at least 90% sequence identity to the amino acid sequence set forth in SEQ ID NO:1 and which comprises an alanine residue at the positions corresponding to positions 263 and 265 of SEQ ID NO:1, wherein said amino acid sequence encodes a thrombin mutant that binds to said thrombin inhibitor;
d) the amino acid sequence of thrombin mutant W215A set forth in SEQ ID NO:2;
e) an amino acid sequence having at least 80% sequence identity to the amino acid sequence set forth in SEQ ID NO:2 and which comprises an alanine residue at the position corresponding to position 263 of SEQ ID NO:2, wherein said amino acid sequence encodes a thrombin mutant that binds to said thrombin inhibitor; and
f) an amino acid sequence having at least 90% sequence identity to the amino acid sequence set forth in SEQ ID NO:2 and which comprises an alanine residue at the position corresponding to position 263 of SEQ ID NO:2, wherein said amino acid sequence encodes a thrombin mutant that binds to said thrombin inhibitor.

23. The method of claim 22, wherein said thrombin mutant comprises the amino acid sequence set forth in SEQ ID NO:1.

24. The method of claim 22, wherein said thrombin mutant comprises the amino acid sequence set forth in SEQ ID NO:2.

25. The method of claim 22, wherein said direct thrombin inhibitor is selected from the group consisting of argatroban or derivatives or analogs thereof, hirudin or recombinant or synthetic derivatives or analogs thereof, a derivative of the tripeptide Phe-Pro-Arg, a chloromethylketone derivative, ximelagatran or derivatives, metabolites, or analogs thereof, an anion binding exosite inhibitor, and an RNA/DNA aptamer.

26. The method of claim 25, wherein said direct thrombin inhibitor is argatroban.

27. The method of claim 25, wherein said recombinant or synthetic derivative or analog of hirudin is selected from the group consisting of bivalirudin, lepirudin, and desirudin.

28. The method of claim 25, wherein said metabolite of ximelagatran is melagatran.


SEQUENCE LISTING (3 sequences)

SEQ ID NO: 1 - protein, 295 aa, Homo sapiens
TFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSAGAGCDRDGKYGFYTHVFRLKKWIQKVIDQFGE

SEQ ID NO: 2 - protein, 295 aa, Homo sapiens
TFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSAGEGCDRDGKYGFYTHVFRLKKWIQKVIDQFGE

SEQ ID NO: 3 - protein, 295 aa, Homo sapiens
TFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE